(19) United States
(12) Patent Application Publication
(10) Pub. No.: US 2026/0091124 A1
(43) Pub. Date: Apr. 2, 2026

(54) CLICK-TO-RELEASE ON PROTEINS AND PEPTIDES

(71) Applicant: VALANX Biotech GmbH, Wien (AT)

(72) Inventor: Michael LUKESCH, Wien (AT)

(21) Appl. No.: 19/113,365

(22) PCT Filed: Sep. 20, 2023
(86) PCT No.: PCT/EP2023/075888

§ 371 Date: Mar. 19, 2025

(30) Foreign Application Priority Data
Sep. 20, 2022 (EP) 22196526.2

(51) Int. Cl. A61K 47/68; C07K 16/30

(52) U.S. Cl. CPC A61K 47/6851; A61K 47/6803; C07K 16/3092; C07K 2317/569

(57) ABSTRACT

The invention relates to a combination of a modified peptide or protein comprising one or more bioorthogonal functional group, and a drug which is modified with one or more bioorthogonal functional group which is complementary to the bioorthogonal group of the peptide or protein.

### FIELD OF THE INVENTION

The invention relates to the combination of a modified drug and a peptide or protein comprising one or more bioorthogonal functional group for bioorthogonal delivery of the drug in a subject in need thereof.

### BACKGROUND ART

Click chemistry has emerged as a versatile tool for in-vivo chemistry: “Click chemistry refers to a group of reactions that are fast, simple to use, easy to purify, versatile, regiospecific, and give high product yields” [1]; these reactions are therefore ideal tools for targeting and labelling biological targets in-vivo. The inverse-electron demand Diels-Alder (iEDDA) reaction between the diene tetrazine and dienophiles shows exceptional kinetics (1-10⁶ M⁻¹s⁻¹) and is, due to its speed and the metabolic stability of some developed reactants especially suited for such in-vivo applications.

Because of its speed and specificity this reaction has been used in the targeted release of caged prodrugs. In this approach, a biologically active molecule is caged (i.e., modified) with a chemical group active in iEDDA chemistry. This modification alters (diminishes) the biological activity of the drug and therefore also its systemic side effects. The biological target, where the drug then should be active in the living organism, is then targeted with the iEDDA reaction counterpart. Upon successful targeting, the caged prodrug is administered, reacts with the iEDDA active counterpart already present in the organism and is released to its full biological activity only upon reacting under elimination of the caging group

Current literature describes various methods of marking the biological target with an iEDDA-reactive moiety, e.g., using local injections [2].

Here, an iEDDA-reactive polymer is directly injected into tumorous tissue. After association of the polymer with the tumor, an iEDDA-reactive prodrug is administered, which is released upon contact with the tumor-associated polymer. This achieves high local concentration of the active drug with reduced systemic toxicity (see FIG. 2). The disadvantage in this approach is that the procedure is invasive, and the tumour needs to be precisely localized before the polymer can be applied.

Alternatively, endogenous chemically reactive markers are used [3]. Some diseases by causing oxidative stress produce acrolein as a by-product of their metabolism. Acrolein is a compound active in 1,3-dipolar cycloadditions, which can also lead to elimination of a chemical group and subsequent drug release from the prodrug. This means that a prodrug is preferentially activated at the disease site where oxidative stress takes place (see FIG. 3). As elegant as this approach is, it suffers from the drawbacks of disease diversity. To be properly efficacious, this potential treatment needs to be administered to disease forms with very high local acrolein concentrations, which needs to be analytically determined beforehand. Inevitably there will be some patient subpopulations no responding due to metabolic diversity of disease forms.

Additionally, protein conjugates may be used [4]. This approach uses proteins modified with an iEDDA-reactive moiety. The protein has an affinity to a surface marker of the biological target to be treated with a drug. The protein conjugate is administered and left to associate with the target. After a suitable time for proper target labelling has passed, the treatment is completed by chasing with an iEDDA-caged prodrug. Upon contacting with the pre-labelled target, the prodrug reacts and the active drug is released (see FIG. 4).

The drawback of this approach is the often-tedious development of protein conjugates, where a lot of resources need to be dedicated to developing a suitable supply chain and analytical methods to verify drug product quality. Conjugation of the iEDDA-reactive moiety needs to be site-specific to yield a reproducible drug molecule and access to such technology is not always possible.

WO2014081301A1 discloses the combination of a masking moiety linked to a trigger moiety which is further linked to a drug. The trigger moiety comprises a dienophile and the activator comprises a diene. The trigger moiety and the activator undergo a fast, bio-orthogonal reaction resulting in the release of the masking moiety and in the activation of the drug. WO2017044983A1 describes bioorthogonal compositions for delivering agents in a subject. The bioorthogonal compositions include a hydrogel support composition having different bioorthogonal functional groups. WO2022032191A1 discloses trans-cyclooctene bioorthogonal agents and their use in cancer and immunotherapy.

Fairhall J. M. et al. disclose the conjugation of functionalized trans-cyclooctenes to cetuximab, providing a reagent for pre-targeting and localization of the bioorthogonal reagent [5].

The drawback of the state-of-the-art conjugates is the often-tedious development of protein conjugates, where a lot of resources need to be dedicated to developing a suitable supply chain and analytical methods to verify drug product quality. Conjugation of the iEDDA-reactive moiety needs to be site-specific to yield a reproducible drug molecule and access to such technology is not always possible.

Thus, there is still a need for a new approach to treat diseases based on modified entities bearing bioorthogonal functional groups.

### SUMMARY OF INVENTION

It is an object of the present invention to provide a novel approach to treat diseases based on modified entities bearing bioorthogonal functional groups. The object is solved by the subject-matter of the present invention. The novel approach is based on the combination of a modified peptide or protein comprising one or more bioorthogonal functional group, and a drug which is modified with one or more bioorthogonal functional group which is complementary to the bioorthogonal group of the protein or peptide.

The present invention relates to a combination of (i) a modified peptide or protein comprising one or more bioorthogonal functional group, and (ii) a drug which is modified with one or more bioorthogonal functional group which is complementary to the bioorthogonal group of (i).

The bioorthogonal functional group may be a dienophile or a diene. The dienophile is for example a trans-cyclooctene. The diene is for example a tetrazine moiety.

According to one embodiment of the invention, the modified peptide or protein is selected from the group consisting of antibodies, antibody fragments, diabodies, single chain variable fragment antibodies, single domain antibodies, nanobodies, small protein binders, carrier proteins, any peptide or protein with affinity to a human disease target.

An “antibody fragment” comprises a portion of an intact antibody, including the antigen binding and/or the variable region of the intact antibody. Examples of antibody fragments include Fab, Fab′, F(ab′)2, and Fv fragments; linear antibodies; single-chain antibody molecules; multivalent single domain antibodies; and multispecific antibodies formed from antibody fragments.

According to one embodiment of the invention, the modified peptide or protein bears at least one diene moiety of general formula (I),

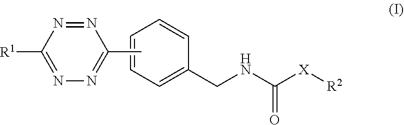

wherein
X denotes NH or O, and
R is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NR^{a}R^{a}, —SR^{a},
R² is an amino acid residue which connects to the next residues towards the N- and C-terminus of the protein or peptide; and
R^{a} is hydrogen or C₁₋₆alkyl.

According to one embodiment of the invention, the drug may be conjugated to dienophile moiety. The drug may be conjugated to the dienophile moiety via a carbamate moiety.

A further embodiment relates to the combination as described herein, wherein the dienophile moiety is a trans-cyclooctene moiety.

A further embodiment relates to the combination as described herein, wherein the drug is selected from the group consisting of cytotoxins, antiproliferative agents, antitumor agents, antiviral agents, antibiotics, anti-inflammatory agents, chemo sensitizing agents, radio sensitizing agents, immunosuppressants, immunostimulants, immunomodulators, anti-angiogenic factors, DNA damaging agents, DNA crosslinkers, DNA binders, DNA alkylators, DNA intercalators, DNA cleavers, microtubule stabilizing and destabilizing agents, and topoisomerases inhibitors.

The drug may is selected from the group consisting of colchinine, vinca alkaloids, anthracyclines, doxorubicin, epirubicin, idarubicin, daunorubicin, camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, dactinomycines, duocarmycins, maytansines, maytansinoids, dolastatins, auristatins, pyrrolobenzodiazepines and dimers, indolinobenzodiazepines and dimers, pyridinobenzodiazepines and dimers, mitomycins, melphalan, leurosine, leurosideine, actinomycin, tallysomycin, lexitropsins, bleomycins, podophyllotoxins, etoposide, etoposide phosphate, staurosporin, esperamicin, the pteridine family of drugs, platinum-based drugs, and cytotoxic nucleosides.

One embodiment of the invention relates to the combination as described herein for use in the treatment of cancer, of infectious disease, or of an autoimmune disease.

A further embodiment relates to the combination as described herein, wherein the cancer is a melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma Wilms tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

A further embodiment relates to the combination as described herein, wherein the modified peptide or protein and the drug are administered sequentially or concomitantly.

### BRIEF DESCRIPTION OF DRAWINGS

FIG. 1: iEDDA reaction of a tetrazine with a dienophile.

FIG. 2: Drug release via pre-targeting with iEDDA-reactive polymer.

FIG. 3: Using acrolein as a reaction partner for in-vivo drug release.

FIG. 4: Using protein conjugates for click-to-release chemistry of highly active toxins in treatment of tumors.

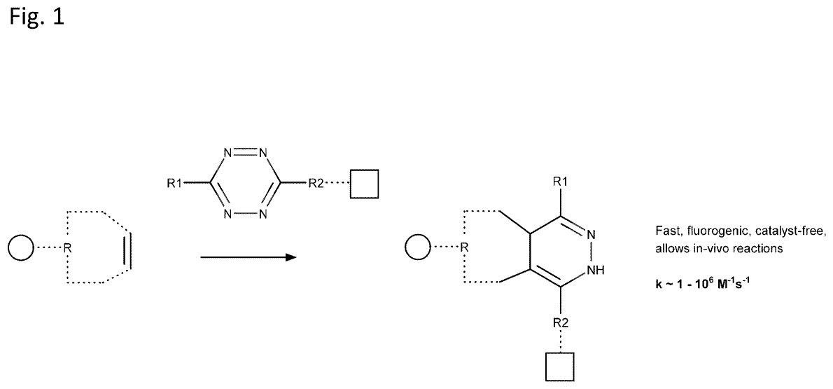

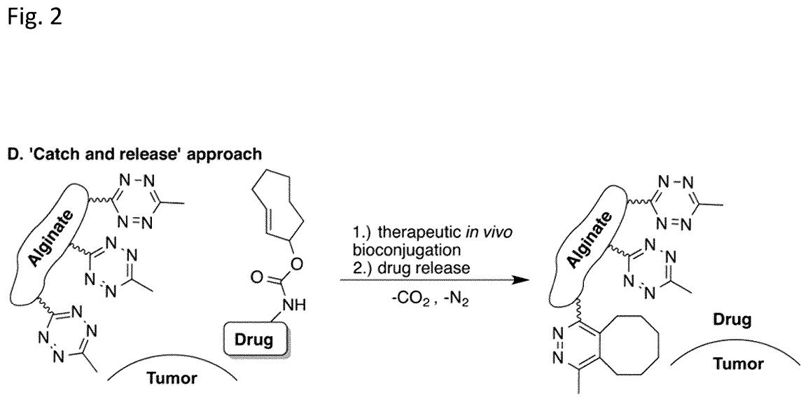

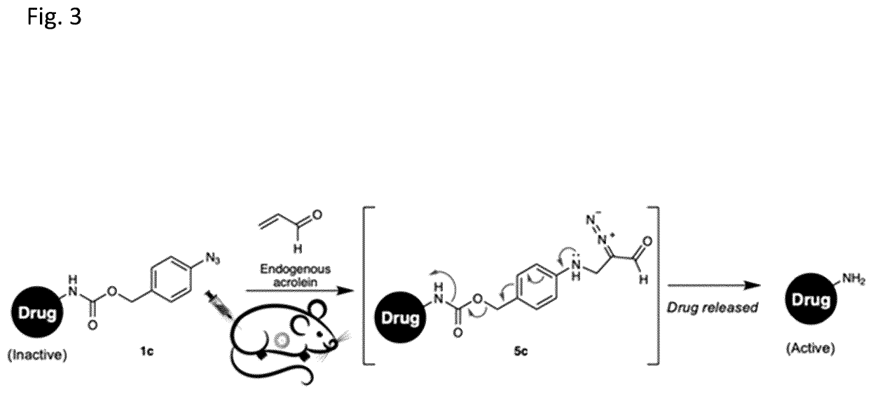

### DESCRIPTION OF EMBODIMENTS

The present invention relates to a combination of (i) a modified peptide or protein comprising one or more bioorthogonal functional group, and (ii) a drug which is modified with one or more bioorthogonal functional group which is complementary to the bioorthogonal group of (i).

In chemistry Diels-Alder reaction is an important reaction in which a conjugated diene reacts with dienophile (a substituted alkene), producing a substituted cyclohexene derivative. This reaction has two partners reacting with each other: the diene and the dienophile.

A diene is unsaturated hydrocarbon consisting of two double bonds between carbon atoms. A diene is also known as diolefin or alkadiene. It is a covalent compound containing two alkene units. Dienes usually exist as subunits of more complex organic molecules. Moreover, dienes can be found in naturally occurring compounds as well as in synthetic chemicals. These chemicals are useful in organic synthesis reactions.

A dienophile is an organic compound that readily reacts with a diene. A dienophile is commonly used in the Diels-Alder reaction that involves the reaction between a conjugated diene and a substituted alkene, wherein the substituted alkene acts as the dienophile.

A proper dienophile typically bears one or two of the following functional groups: CHO, COR, COOR, CN, C═C, Ph, or halogen. Moreover, the diene has to be highly electron-rich.

An alkene is usually known as a dienophile because it reacts with a diene readily. Typically, we do not need heat in Diels-alder reactions, but heating can improve the yield of the reaction.

The term bioorthogonal chemistry refers to any chemical reaction that can occur inside of living systems without interfering with native biochemical processes. The concept of the bioorthogonal reaction has enabled the study of biomolecules such as glycans, proteins, and lipids in real time in living systems without cellular toxicity. A number of chemical ligation strategies have been developed that fulfill the requirements of bioorthogonality, including the 1,3-dipolar cycloaddition between azides and cyclooctynes (also termed copper-free click chemistry), and the tetrazine ligation.

In order to be considered bioorthogonal, a reaction must fulfill a number of requirements:
Selectivity: The reaction must be selective between endogenous functional groups to avoid side reactions with biological compounds;
Biological inertness: Reactive partners and resulting linkage should not possess any mode of reactivity capable of disrupting the native chemical functionality of the organism under study;
Chemical inertness: The covalent link should be strong and inert to biological reactions;
Kinetics: The reaction must be rapid so that covalent ligation is achieved prior to probe metabolism and clearance. The reaction must be fast, on the time scale of cellular processes (minutes) to prevent competition in reactions which may diminish the small signals of less abundant species. Rapid reactions also offer a fast response, necessary in order to accurately track dynamic processes;
Reaction biocompatibility: Reactions have to be non-toxic and must function in biological conditions taking into account pH, aqueous environments, and temperature. Pharmacokinetics are a growing concern as bioorthogonal chemistry expands to live animal models;
Accessible engineering: The chemical reporter must be capable of incorporation into biomolecules via some form of metabolic or protein engineering. Optimally, one of the functional groups is also very small so that it does not disturb native behavior.

According to the invention, a peptide or protein may be modified by one or more bioorthogonal functional groups. The peptide or protein may either bear a diene or a dienophile. For example, the protein may be modified by incorporating a diene moiety, e.g., an amino acid bearing a tetrazine moiety.

Such amino acid compounds bearing a tetrazine moiety are of general formula (I),

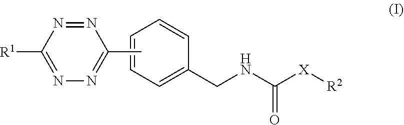

wherein
X denotes N or O, and
R is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NR^{a}R^{a}, —SR^{a},
R² is an amino acid residue which connects to the next residues towards the N- and C-terminus of the protein or peptide; and
R^{a} is hydrogen or C₁₋₆alkyl.

Methods for producing such modified amino acids are described in international patent application PCT/EP2022/064273.

Preferably, the tetrazine moiety is incorporated at predefined sites of the peptide or protein. The accordingly modified peptide or protein may comprise one single amino acid or multiple amino acids bearing the tetrazine moiety. Having amino acids bearing a tetrazine moiety at predefined sites provides the ability to produce a precisely defined peptide or protein.

Some embodiments of the invention relate to methods of producing a peptide or protein comprising a single or multiple tetrazine moieties, said methods comprising genetically incorporating a synthetic amino acid comprising a tetrazine moiety into a peptide or protein. Genetically incorporating the tetrazine moiety allows precise construction of a defined peptide or protein conjugate. The location of the tetrazine moieties can be precisely controlled. This advantageously avoids the need to subject the whole peptide or protein to complex reaction steps using chemical functional groups occurring in the natural amino acids.

Suitably the method described for producing the peptide or protein comprises
(i) providing a nucleic acid encoding the peptide or protein which nucleic acid comprises an orthogonal codon encoding the amino acids having a tetrazine moiety;
(ii) translating said nucleic acid in the presence of an orthogonal tRNA synthetase/tRNA pair capable of recognizing said orthogonal codon and incorporating said amino acid having a tetrazine moiety into the peptide or protein chain. Suitably said orthogonal codon comprises an amber codon (TAG), said tRNA comprises tRNAcuA and said tRNA synthetase comprises PyIRS from the organisms Methanosarcina mazei/Methanosarcina bakeri/Methanomethylophilus alvus

In other embodiments the peptide or protein comprises a dienophile moiety. The dienophile moiety may be selected from spirohexene, vinylboronic acids, norbornene, cyclopropene derivatives, cyclooctyne and trans-cyclooctene (TCO). Preferably, trans-cyclooctene is used as dienophile.

The accordingly modified proteins and peptides can be evolved to have affinity to any biological target that marks disease.

According to one embodiment of the invention, a drug is modified with one or more functional group. The functional group is either a diene or a dienophile and which is complementary to the functional group used in the modified peptide or protein. Thus, in case the peptide or protein is modified with a diene, the drug is modified with a dienophile. In case the peptide or protein is modified with a dienophile, then the drug is modified with a diene.

The term “drug” refers to an agent capable of treating and/or ameliorating a condition or disease, or one or more symptoms thereof, in a subject. Drugs of the present disclosure also include prodrug forms of therapeutic agents.

The drug may be selected from the group consisting of cytotoxins, antiproliferative agents, antitumor agents, antiviral agents, antibiotics, anti-inflammatory agents, chemo sensitizing agents, radio sensitizing agents, immunosuppressants, immunostimulants, immunomodulators, anti-angiogenic factors, DNA damaging agents, DNA crosslinkers, DNA binders, DNA alkylators, DNA intercalators, DNA cleavers, microtubule stabilizing and destabilizing agents, and topoisomerases inhibitors.

For example, the drug is selected from the group consisting of colchinine, vinca alkaloids, anthracyclines, doxorubicin, epirubicin, idarubicin, daunorubicin, camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, dactinomycines, duocarmycins, maytansines, maytansinoids, dolastatins, auristatins, pyrrolobenzodiazepines and dimers, indolinobenzodiazepines and dimers, pyridinobenzodiazepines and dimers, mitomycins, melphalan, leurosine, leurosideine, actinomycin, tallysomycin, lexitropsins, bleomycins, podophyllotoxins, etoposide, etoposide phosphate, staurosporin, esperamicin, the pteridine family of drugs, platinum-based drugs, and cytotoxic nucleosides.

The combination of an accordingly modified peptide and protein and of a drug may be used in treatment and/or diagnosis of a condition or disease in a subject that is amenable to treatment or diagnosis by administration of the modified drug.

The combination as described herein may be used in the treatment of cancer, of infectious disease, or of an autoimmune disease.

In certain embodiments, the combination as described herein may be used for the treatment of cancer. The cancer may be a melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma Wilms tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

By incorporating one or multiple synthetic amino acids into proteins and peptides that are active in iEDDA-reactions, we provide a way to solve the above-mentioned drawbacks of the various approaches of targeted drug release for treating various diseases. This enables us to evolve treatments for various indications. By avoiding the development of conjugates and use the protein or peptide directly for click-to-release reactions we simplify the development of such therapeutics, relying on established protein purification schemes without the additional burden of protein conjugate development. The efficacy and release activity of the targeting protein or peptide can be easily tuned by incorporating multiple synthetic amino acids, which in turn releases more drug at the targeted site.

### Methods:

Incorporation of a Synthetic Amino Acid Modified with Tetrazine into a Protein:

A mutant pyrrolysyl-tRNA synthetase, obtained from a wild-type pyrrolysyl-tRNA synthetase, which is Methanosarcina, Methanocaldococcus, Methanomethylophilus or other derived pyrrolysyl-tRNA synthetase, and/or the mutant pyrrolysyl-tRNA synthetase aminoacylates a pyrrolysine tRNA, incorporates modified amino acids as described herein into proteins and peptides.

Incorporation of the Tetrazine Amino Acid into a Nanobody Protein and Subsequent Active Drug Release

A mutant pyrrolysyl-tRNA synthetase, obtained from a wild-type pyrrolysyl-tRNA synthetase, which is an archaeal-derived pyrrolysyl-tRNA synthetase (such as Methanosarcina or Methanocaldococcus or Methanomethylophilus or other), and/or the mutant pyrrolysyl-tRNA synthetase aminoacylates a pyrrolysine tRNA, incorporates amino acids as described herein. The mutant pyrrolysyl-tRNA synthetase was generated by state-of-the-art protein engineering technologies, such as structure guided site-saturation mutagenesis or directed evolution or a combination. Also, other technologies such as gene shuffling would be possible.

The mutant pyrrolysyl-tRNA synthetase and the corresponding amber suppressor pyrrolysine tRNA were introduced into an expression vector harboring a pBR322 origin of replication, a nanobody protein carrying an in-frame amber stop codon at amino acid position 65, as well as a C-terminal hexahistidine tag and a kanamycin resistance gene. The mutant pyrrolysyl-tRNA synthetase was expressed from an inducible promoter and the suppressor pyrrolysine tRNA from a constitutive promoter commonly used for this purpose.

E. coli cells harboring the above-described expression vector were cultivated in 250 mL flasks each containing 50 mL M9 minimal medium with 1-2% glucose as C-source or standard 2×YT medium with 50 μg/mL kanamycin (Roth). Cultures were incubated at 37° C. on an orbital shaker at 160-180 rpm. At D600 of 0.8-1.0, the expression of the PyIRS was induced by adding 0.2% (w/v) of inducer (Roth). In addition, 0.1-10 mM of tetrazine-lysine dissolved in 0.1 M HCl or DMSO or H₂O or a mixture of the previous. Expression was carried out between 4-24 hours (temperature can be adjusted depending on the target protein; 37° C. for the nanobody). Cells were harvested by centrifugation (5,000 g for 30 minutes at 4° C.). The nanobody variant was purified by Ni2+-affinity chromatography using Ni-NTA agarose following the instructions of the manufacturer.

Purified nanobody variant carrying the tetrazine-lysine was contacted in solution with trans-cyclooctene-(TCO)-doxorubicine, a prodrug of doxorubicine. This lead to reaction of TCO-doxorubicine with the tetrazine-lysine in the nanobody and subsequent release by elimination of the active drug doxorubicine.

The presence and release of doxorubicin was confirmed by High-performance-liquid-chromatography coupled to a mass spectrometer employing the appropriate standards. The release of doxorubicin directly on the nanobody was confirmed with these measurements.

### REFERENCES

[1] D. Hein, Christopher. Liu, Xin-Ming. Wang, D. Click Chemistry, a Powerful Tool for Pharmaceutical Sciences. Natl. Inst. Heal. J. 25, 1-7 (2008).
   [2] Oneto, J. M. M., Khan, I., Seebald, L. & Royzen, M. In vivo bioorthogonal chemistry enables local hydrogel and systemic pro-drug to treat soft tissue sarcoma. ACS Cent. Sci. 2, 476-482 (2016).
[3] Pradipta, A. R. et al. Targeted 1,3-dipolar cycloaddition with acrolein for cancer prodrug activation. Chem. Sci. 12, 5438-5449 (2021).
[4] Rossin, R. et al. Chemically triggered drug release from an antibody-drug conjugate leads to potent antitumour activity in mice. Nat. Commun. 9, 1-11 (2018).
[5] Fairhal, J. M. et al., EGFR-targeted prodrug activation using bioorthogonal alkene-azide click-and-release chemistry. Bioorg. Med. Chem. 46, 1-11 (2021).

1. A composition comprising a combination of:
a. a modified peptide or protein comprising one or more bioorthogonal functional groups, and
b. a drug which is modified with one or more bioorthogonal functional groups which is reactive with the one or more bioorthogonal groups of (a),
wherein the one or more bioorthogonal functional groups of (b) are eliminated when contacted with the one or more bioorthogonal functional groups of (a), thereby leading to the release of unmodified drug.

2. The composition of claim 1, wherein the one or more bioorthogonal functional groups comprise a dienophile or a diene.

3. The composition of claim 2, wherein the dienophile is a trans-cyclooctene dienophile.

4. The composition of claim 3, wherein the diene is a tetrazine moiety.

5. The composition of claim 1, wherein the modified peptide or protein is selected from the group consisting of antibodies, antibody fragments, diabodies, single chain variable fragment antibodies, single domain antibodies, nanobodies, protein binders, carrier proteins, and any peptide or protein with affinity to a human disease target.

6. The composition of claim 1, wherein the modified peptide or protein bears at least one diene moiety of general formula (I), 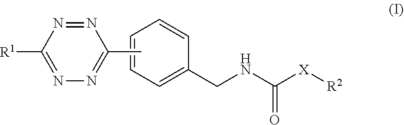
wherein
X denotes NH or O;
R¹ is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NRaRa, —SR^{a};
R² is an amino acid residue which connects to the next residues towards the N- and C-terminus of the protein or peptide; and
R^{a} is hydrogen or C₁₋₆alkyl.

7. The composition of claim 1, wherein the drug is conjugated to a dienophile moiety.

8. The composition of claim 7, wherein the drug is conjugated to the dienophile moiety via a carbamate moiety.

9. The composition of claim 7, wherein the dienophile moiety is a trans-cyclooctene moiety.

10. The composition of claim 1, wherein the drug is selected from the group consisting of cytotoxins, antiproliferative agents, antitumor agents, antiviral agents, antibiotics, anti-inflammatory agents, chemo sensitizing agents, radio sensitizing agents, immunosuppressants, immunostimulants, immunomodulators, anti-angiogenic factors, DNA damaging agents, DNA crosslinkers, DNA binders, DNA alkylators, DNA intercalators, DNA cleavers, microtubule stabilizing and destabilizing agents, and topoisomerases inhibitors.

11. The composition of claim 10, wherein the drug is selected from the group consisting of colchinine, vinca alkaloids, anthracyclines, doxorubicin, epirubicin, idarubicin, daunorubicin, camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, dactinomycines, duocarmycins, maytansines, maytansinoids, dolastatins, auristatins, pyrrolobenzodiazepines and dimers, indolinobenzodiazepines and dimers, pyridinobenzodiazepines and dimers, mitomycins, melphalan, leurosine, leurosideine, actinomycin, tallysomycin, lexitropsins, bleomycins, podophyllotoxins, etoposide, etoposide phosphate, staurosporin, esperamicin, the pteridine family of drugs, platinum-based drugs, and cytotoxic nucleosides.

12. A method of treating cancer, an infectious disease, or an autoimmune disease comprising the step of administering a therapeutically effective amount of the composition of claim 1 to a subject in need thereof.

13. The method of claim 12, wherein the cancer is a melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma Wilms tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

14. The method of claim 12, wherein the modified peptide or protein and the drug are administered sequentially or concomitantly.
